(19) United States
(12) Patent Application Publication
(10) Pub. No.: US 2026/0256468 A1
(43) Pub. Date: Sep. 3, 2026

(54) FULLY CANULATED SOFT TISSUE TO BONE ANCHOR AND METHOD

(71) Applicant: SHOULDER SOLUTIONS CORPORATION, Tempe, AZ (US)

(72) Inventor: Sumit Dewanjee, Tempe, AZ (US)

(73) Assignee: SHOULDER SOLUTIONS CORPORATION, Tempe, AZ (US)

(21) Appl. No.: 19/100,465

(22) PCT Filed: Jul. 31, 2023
(86) PCT No.: PCT/US2023/029155

§ 371 Date: Jan. 31, 2025

Related U.S. Application Data
(60) Provisional application No. 63/393,962, filed on Jul. 31, 2022.

(51) Int. Cl. A61B 17/04

(52) U.S. Cl. CPC A61B 17/0401; A61B 2017/044; A61B 2017/0445

(57) ABSTRACT

An implant to repair soft tissue to bone using an implant with a k-wire (Kirschner wire) guide system is described. The implant is cannulated for anchoring a suture and includes: a tip having a tip k-wire passage and at least one tip suture passage and a screw having a screw k-wire passage, wherein the tip k-wire passage and the screw k-wire passage are aligned. Optionally, the implant may include at least one disc having a disc k-wire passage and at least one disc suture passage for insertion between the tip and the screw.

This application claims priority of U.S. Provisional Patent Application No. 63/393,962 having a filing date of Jul. 31, 2022, and U.S. Provisional Patent Application No. 63/393,962 is hereby incorporated by reference in its entirety, including any appendices, in this patent application as if fully set forth herein.

### FIELD

The present invention relates to an implant used to repair soft tissue; for example, cartilage, ligament(s) or tendon(s), to the bone using a cannulated implant with a k-wire (Kirschner wire) guide system providing increased precision and ease of deployment. It is proposed that such an implant and method can improve surgical outcome and efficiency such as reducing operating time and/or loss. Additionally, the implant may have one or more cannulated disc(s) for additional suture placements.

### BACKGROUND

K-wires are widely used in orthopedics and other types of medical and veterinary surgery. K-wires come in different sizes and are used to hold bone fragments together or to provide an anchor for skeletal traction. K-wires are used for temporary fixation during some operations. After definitive fixation they are then removed.

Implants to repair soft tissue to bone are known, but are not cannulated for use with a k-wire for guidance.

The following background patents and patent publications, are incorporated by reference in their entireties, are disclosed merely for background purposes and relevant to the state of the art, but do not contain one or more of the elements of the present invention: U.S. Pat. No. 10,881,388 B2, US 20200170634 A1, US 20180271514 A1, US 20170209139 A1, US 20140277134 A1, US 20120150226 A1, US 20120150225 A1, US 20090318965 A1, US 20080004659 A1, US 20070142861 A1, US 20050283156 A1, US 20040106950 A1, US 20040093031 A1, US 20030004545 A1, and U.S. Pat. No. 6,267,766 B1.

Some of the primary benefits possibly include improved accuracy of placement, decreased operating room time by saving time in placement, and preserved bone with ability to substitute a larger implant or its parts, such as a larger screw in osteoporotic bone as well as ease of implant removal in case of intra-operative need to adjust position throughout soft tissue (e.g. tendons) of implant. Additionally, stackable disc(s) are disclosed that allow additional suture points. The present invention can provide additional suture points to distribute forces more evenly and reduce possible tearing in the soft tissue repair, such as tendon repair. The present invention intends to improve outcomes, improve safety and decrease costs for tendon to bone repair.

### SUMMARY

The structure, overall operation and technical characteristics of the present invention will become apparent with the detailed description of preferred embodiments and the illustrations of the related drawings herein.

A preferred embodiment of the invention includes an implant that is cannulated for anchoring a suture and used with a k-wire including a tip having a tip front and a tip k-wire passage and at least one tip suture passage separated from the tip k-wire passage, and a screw having a screw back and a screw k-wire passage, wherein the tip k-wire passage and the screw k-wire passage are aligned and wherein the tip front is an implant front and the screw back is the implant back. Preferably, the tip includes a tip central axis and the screw includes a screw central axis wherein the tip k-wire passage is centered on the tip central axis and the screw k-wire passage is centered on the screw central axis.

Also preferably, each at least one tip suture passage includes a tip suture entrance and a tip suture exit wherein the tip suture passage is offset from the tip k-wire passage. More preferably, the tip suture passage entrance of each at least one tip suture passage is selected from the group consisting of: the tip suture entrance extending to a portion of the front of the tip and the tip suture entrance not extending to the front of the tip, wherein the tip suture passage exit of each at least one tip suture passage is selected from the group consisting of: the tip suture exit extending to a portion of the front of the tip and the tip suture exit not extending to the front of the tip.

In another preferred embodiment, the tip and the screw are separate and the tip has a tip back and the screw has a screw front wherein the implant from the implant front to the implant back includes, in order, the tip front, the tip back, the screw front and the screw back. In this preferred embodiment, the tip suture passage exit can either extend or not extend to the tip back.

In another embodiment of the invention, the implant may also include at least one disc: each at least one disc having a disc front, a disc back, a disc side, a disc central axis and a disc k-wire passage centered on the disc central axis and at least one disc suture passage separated from the disc k-wire passage and wherein the tip k-wire passage, the disc k-wire passage for each at least one disc, and the screw passage are aligned and wherein the implant from the implant front to the implant back includes, in order, the tip from the tip front to the tip back, each at least one disc each disc from the at least one disc front to the at least one disc back, and the screw from the screw front to the screw back. More preferably, each at least one disc suture passage includes: a disc suture passage entrance selected from the group consisting of: the disc suture passage entrance extending to the disc front, and the disc suture passage entrance extending to the disc side, and the disc suture passage entrance extending to the disc back, and a disc suture passage exit selected from the group consisting of: the disc suture passage exit extending to the disc front, the disc suture passage exit extending to the disc side, and the disc suture passage entrance extending to the disc back. Other preferred embodiments include each the at least one tip suture passage is at least two, each of the at least one tip suture passage is at least three or each the at least one tip suture passage is at least four.

An another embodiment of the invention resides in an implant that is cannulated for anchoring a suture and used with a k-wire comprising: a tip having a tip front, a tip central axis and a tip k-wire passage wherein the tip k-wire passage is centered on the tip central axis, and at least one disc: each at least one disc having a disc front, a disc back, a disc side, a disc central axis and a disc k-wire passage centered on the disc central axis and at least one disc suture passage separated from the disc k-wire passage, and a screw having a screw back, a screw central axis and a screw k-wire passage wherein the screw k-wire passage is centered on the screw central axis, and wherein the tip k-wire passage, the disc k-wire passage for each at least one disc, and the screw passage are aligned, and wherein the tip front is an implant front and the screw back is the implant back, and wherein the implant from the implant front to the implant back includes, in order, the tip from the tip front to the tip back, each at least one disc each disc from the at least one disc front to the at least one disc back, and the screw from the screw front to the screw back. More preferably, each at least one disc suture passage includes: a disc suture passage entrance selected from the group consisting of: the disc suture passage entrance extending to the disc front, and the disc suture passage entrance extending to the disc side, and the disc suture passage entrance extending to the disc back, and a disc suture passage exit selected from the group consisting of: the disc suture passage exit extending to the disc front, the disc suture passage exit extending to the disc side, and the disc suture passage entrance extending to the disc back. Other preferred embodiments include each the at least one disc suture is at least two, each of the at least one disc suture is at least three or each the at least one disc suture is at least four.

Most preferably, each the at least one disc suture passage geometry does not include corners.

Another embodiment of the invention includes a kit used for anchoring a suture using a k-wire, comprising: A drill bit having a cannula for a k-wire and a drill bit central axis and including a punch and a tap wherein the cannula surrounds and is aligned with the drill bit central axis and a k-wire for use with the cannulated drill bit. More preferably, the kit includes an implant as described herein . . .

### BRIEF DESCRIPTION OF THE DRAWINGS

The disclosure is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 illustrates an exploded side view of a preferred embodiment of an implant,

FIG. 2 illustrates an exploded perspective view of the preferred embodiment shown in FIG. 1,

FIG. 3 illustrates an exploded cross-sectional view of the preferred embodiment shown in FIG. 1,

FIG. 4 (a-c) illustrates a front, side and perspective view of the tip in the preferred embodiment shown in FIG. 1,

FIG. 5 illustrates a cross-sectional view of the at least one disc in FIG. 3,

FIG. 6 (a-d) illustrates a side view (a), an end view (b), a back perspective view (c) and a front perspective (d) of the preferred embodiment of the at least one disc of FIG. 1,

FIG. 7 illustrates a cross-sectional view of the screw in FIG. 3,

FIG. 8 (a-d) illustrates a side view (a), an end view (b), a back perspective view (c) and a front perspective (d) of the preferred embodiment of the screw of FIG. 1,

FIG. 9 illustrates a preferred embodiment for a method of use of a k-wire inserted into a bone (the bone is intended to be shown in cross-section),

FIG. 10 illustrates a preferred embodiment of a drill bit,

FIG. 11 illustrates one preferred embodiment for a method of use of a drill bit and a k-wire inserted into a bone (the bone is intended to be shown in cross-section),

FIG. 12 illustrates a second preferred embodiment for a method of use of a drill bit and a k-wire inserted into a bone (the bone is intended to be shown in cross-section),

FIG. 13 illustrates a third preferred embodiment for a method of use of a drill bit and a k-wire inserted into a bone (the bone is intended to be shown in cross-section),

FIG. 14 illustrates preferred embodiment for a method of use of an implant and k-wire inserted into a bone (the bone is intended to be shown in cross-section),

FIG. 15 illustrates preferred embodiment for a method of use of an implant and k-wire inserted into a bone (the bone is intended to be shown in cross-section),

FIG. 16 illustrates preferred embodiment for a method of use of an implant and k-wire inserted into a bone (the bone is intended to be shown in cross-section),

FIG. 17 illustrates preferred embodiment for a method of use of an implant and k-wire inserted into a bone (the bone is intended to be shown in cross-section),

FIG. 18 illustrates preferred embodiment for a method of use of an implant and k-wire inserted into a bone (the bone is intended to be shown in cross-section),

FIG. 19 illustrates preferred embodiment for a method of use of an implant inserted into a bone (the bone is intended to be shown in cross-section), and

FIG. 20 illustrates another preferred embodiment for a method of use of an implant and k-wire inserted into a bone (the bone is intended to be shown in cross-section),

### DETAILED DESCRIPTION

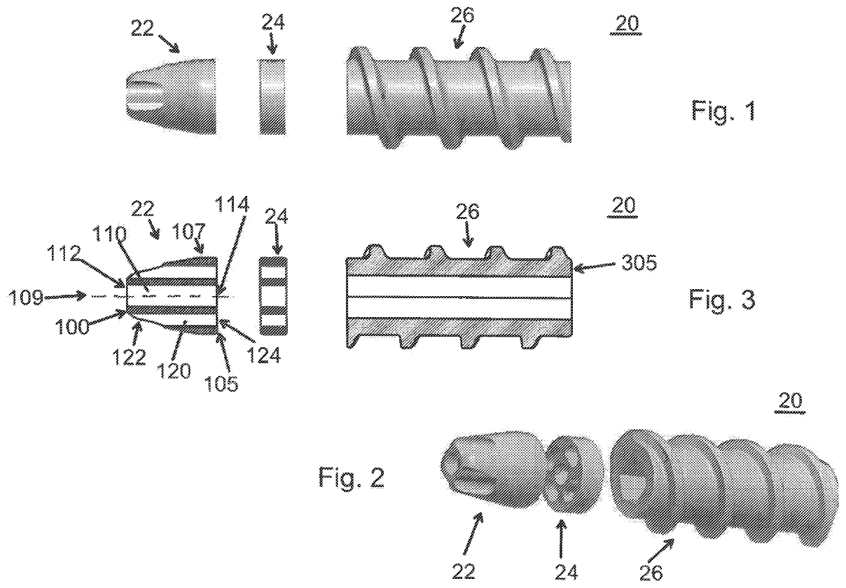

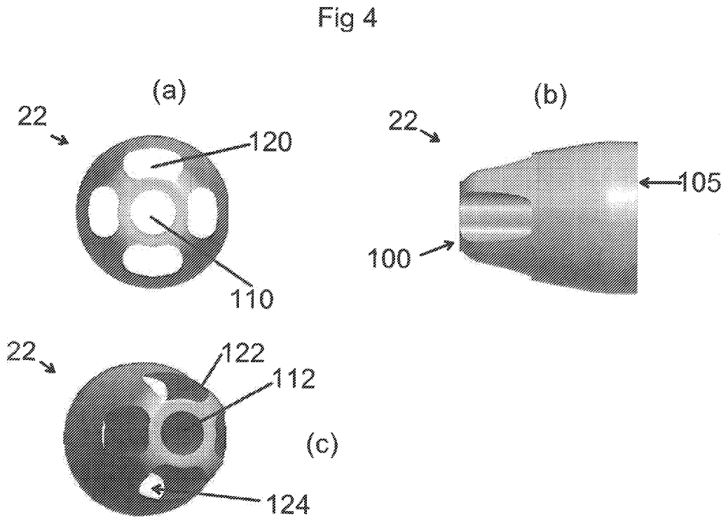

FIGS. 1 and 2 shows an exploded side and perspective views, respectively, of a preferred embodiment of the implant 20 comprising a tip 22, a at least one disc 24 and a screw 26. In this preferred embodiment, the at least one disc shown may be optionally removed or additional disc(s) may be inserted between the tip 22 and the screw 26. Additionally, the implant 20 may only include the tip 22 and the screw 26, and they may be a singular unit, but in other configurations the tip 22 and screw 26 may not necessarily physically fasten together. Additionally, the tip 22 has a tip front 100 and the screw has a screw back 305. FIG. 4 (a-c) shows a side view, an end view and a perspective view a preferred embodiment of the tip 22 shown in FIG. 1.

FIG. 3 shows a cross-sectional view of FIG. 1 showing various internal passages of the tip 22, the at least one disc 24 and the screw 26. In this preferred embodiment, the tip 22 includes a tip back 105, a tip side 107, a tip central axis 107, and a tip k-wire passage 110 surrounds the tip central axis 109 and having a tip k-wire passage entrance 112 and a tip k-wire passage exit 114.

Further, this embodiment in FIG. 2 includes multiple of an at least one tip suture passage(s) 120 each having a tip suture passage entrance 122 and a tip suture passage exit 124. It should be noted that in this embodiment the tip k-wire passage 110 and each of the at least one tip suture passages are entirely separated. However, separation of the passages 110 and 120 need only be enough to prevent a suture from blocking the k-wire movement through the tip k-wire passage 110. It should also be noted that in this preferred embodiment, a portion of each tip suture passage opening(s) 122 extend toward the tip front 100, and each tip suture passage exit(s) 124 extend toward the tip back 105, But in other possible embodiments, one or more of the each tip suture passage opening(s) 122 may not extend toward the tip front 100 and one or more of the each tip suture passage exit(s) 124 may not extend toward the tip back 105; for example, each at least one tip suture passage opening(s) 122 and/or each at least one tip suture exit(s) 124 may open to the tip side 107. Additional examples of other possible embodiments include: (a) one or more of the each tip suture passage opening(s) 122 extending to toward the tip front 100 and each at least one tip suture exit(s) 124 may open to the tip front 100 and (b) one or more of the each tip suture passage opening(s) 122 extending to toward the tip back 105 and each at least one tip suture exit(s) 124 may open to the tip back 105. While the tip 22 may be of the geometry shown in FIGS. 1-4, it is to be understood that the tip 22 may be of any geometry, some examples include, but are not limited to, spherical, cylindrical, and conical.

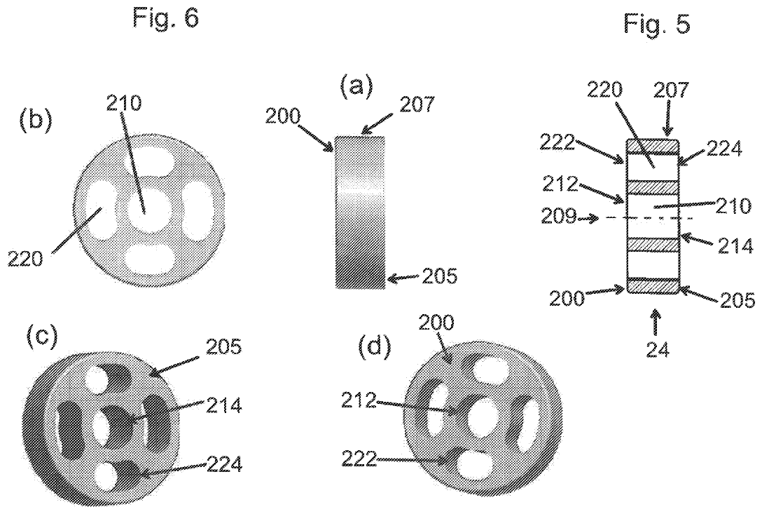

FIG. 5 shows a cross-sectional view of the at least one disc 24 in FIG. 3 showing various internal passages of the at least one disc 24. In this preferred embodiment, the at least one disc 24 includes an at least one disc front 200, an at least one disc back 205, an at least one disc side 207, an at least one disc central axis 209, an at least one disc k-wire passage 210 surrounds the at least one disc central axis 209 and having an at least one disc k-wire entrance 212 and an at least one disc k-wire exit 214. Further, this embodiment in FIG. 5 includes multiple of an at least one disc suture passage(s) 220 each having a disc suture passage entrance 222 and a disc suture passage exit 224. It should be noted that in this embodiment the tip k-wire passage 210 and each of the at least one disc suture passages are entirely separated; however, separation of the passages 210 and 220 need only prevent a suture from blocking the k-wire movement through the at least one disc k-wire passage 210. It should also be noted that in this preferred embodiment, each at least one disc suture passage opening(s) 222 extend toward the each at least one disc suture front 200 and each at least one disc suture passage exit(s) 224 extend toward the tip back 205. But in other possible embodiments, one or more of the each at least one disc suture passage opening(s) 222 may not extend toward the each at least one disc front 200 and one or more of the each at least one disc suture passage exit(s) 224 may not extend toward the each at least one disc suture back 205; for example, each at least one disc suture passage opening(s) 222 and/or each at least one disc suture exit(s) 224 may open to the at least one disc suture side 207. FIG. 6 (a-d) shows a side view (a), an end view (b), a back perspective view (c) and a front perspective (d) of a preferred embodiment of the at least one disc 24 shown in FIG. 1.

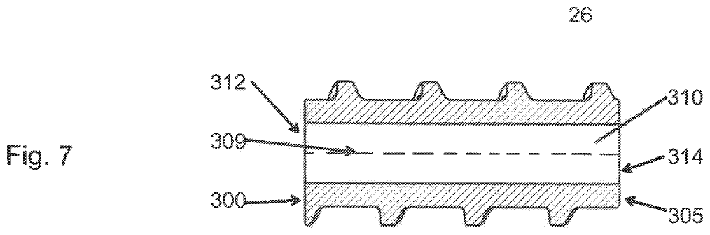

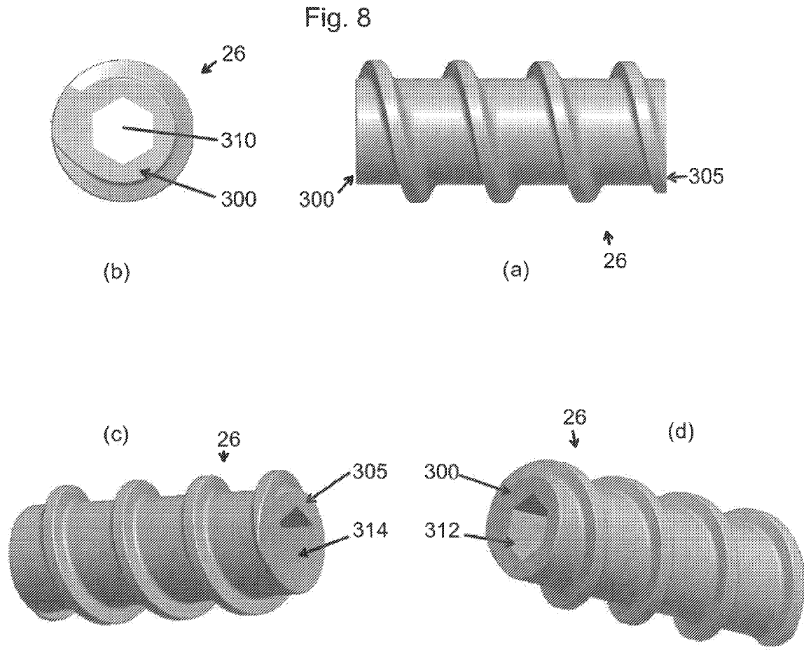

FIG. 7 shows a cross-sectional view of the screw 26 in FIG. 3 showing various internal passages of the screw 26. In this preferred embodiment, the screw 26 includes a screw front 300, the screw back 305, a screw central axis 309, a screw k-wire passage 310 surrounds the screw central axis 309 and having a screw k-wire entrance 312 and a screw k-wire exit 314. Preferably, the screw k-wire passage is configured to couple to a drill bit driver (not shown but may be a screw driver 700); preferably using geometrical coupling arrangement; and more preferably including a drill bit driver k-wire passage (not shown but may be a screw driver k-wire passage 740). FIG. 8 (a-d) shows a side view (a), an end view (b), a back perspective view (c) and a front perspective (d) of a preferred embodiment of the screw 26 shown in FIG. 1. While the screw 26 shown in FIG. 8 is helical, the term screw as used herein may also include other geometries that can be screwed or pressed, such as press fit anchors including those that expand, toggle or have barbs preventing the anchor from backing out.

In the preferred embodiment illustrated in FIGS. 1 and 3, the tip 22, the at least one disc 24 and the screw 26 have each of their respective central axis aligned and are in the following order tip front 100, tip back 105, at least one disc front 200, at least one disc back 205, screw front 300 and screw back 305 to form the implant 20. Other embodiments may remove or add additional at least one disc(s) 24.

In the preferred embodiments, the at least one tip suture passage(s) 120 and the at least one disc suture passage(s) 220 are of any geometrical configuration to allow passage of a suture. Preferably, the at least one tip suture passage(s) and the at least one disc suture passage(s) do not have corners. More preferably, the at least one tip suture passage(s) and the at least one disc suture passage(s) are cylindrical or kidney shaped passages.

In the preferred embodiment in this invention, the tip 22, the disc 24, and the screw 26 do not have the same radius; for example, the screw 26 having the best to affect the best bone to soft tissue repair can be chosen.

Preferably, the implant 20 can be of any biologically compatible material, such as surgical stainless steel, titanium or titanium alloys, metal alloy, biocomposites, sutures and braded sutures; however, the implant is preferably is made of polyether ketone (PEEK).

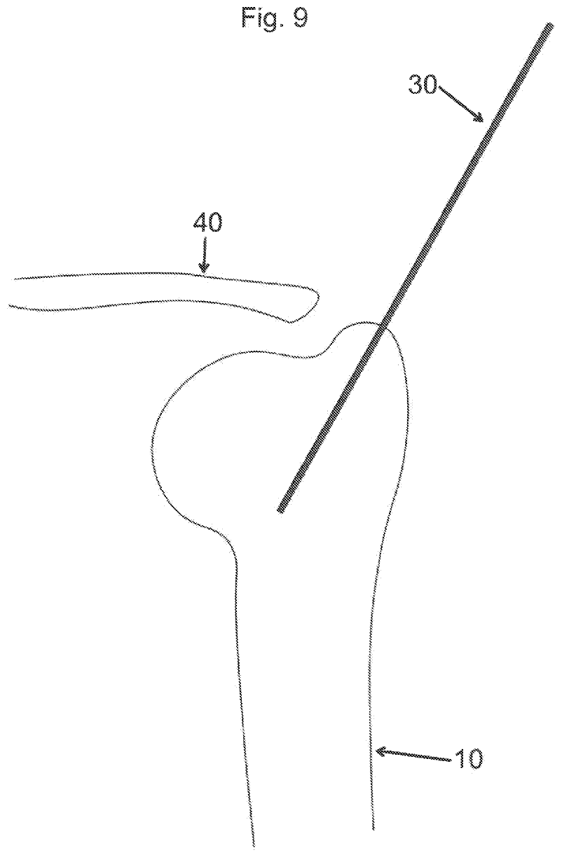

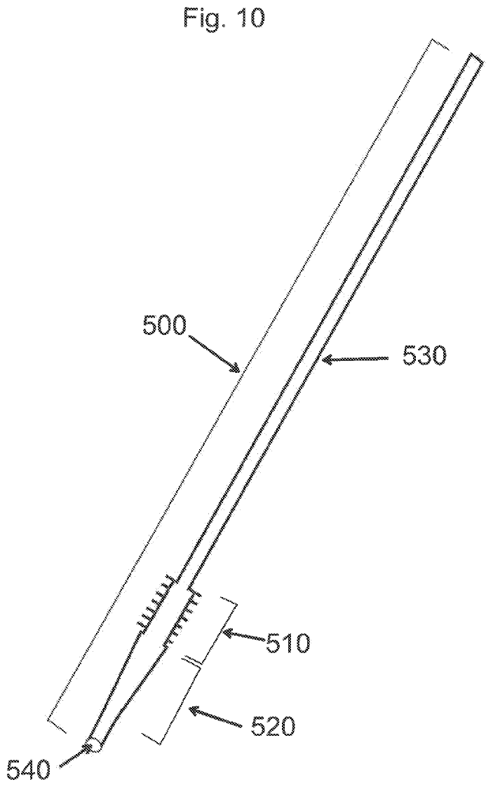

The preferred embodiment for a method of use of the implant 20 is described with reference to FIGS. 9-20. In the preferred embodiment illustrated in FIG. 9, a k-wire 30 is inserted with a drill handle (not shown) into a bone 10 having a detached tendon 40 at a location for the intended tendon 40 repair on the bone 10. In this specific illustration, the bone 10 is the humerus bone, but this method is not limited to tendon repair with the humerus bone and may be used to repair other soft tissues with other bones. It is also envisioned that the implant 20 may also be used to repair one bone to another bone.

As seen in FIG. 10, another preferred embodiment includes a drill bit 500 with an combined punch 520, tap 510, and shank 530. It is to be understood that drill bit 500 is cannulated with a drill bit k-wire passage 540. While this preferred embodiment is combined, it is to be understood that the shank 530, the tap 510 and the punch 520 can be separated so long as they may couple to perform creating opening 90 (not shown in FIG. 10) in the bone 10 configured to receive the implant 20.

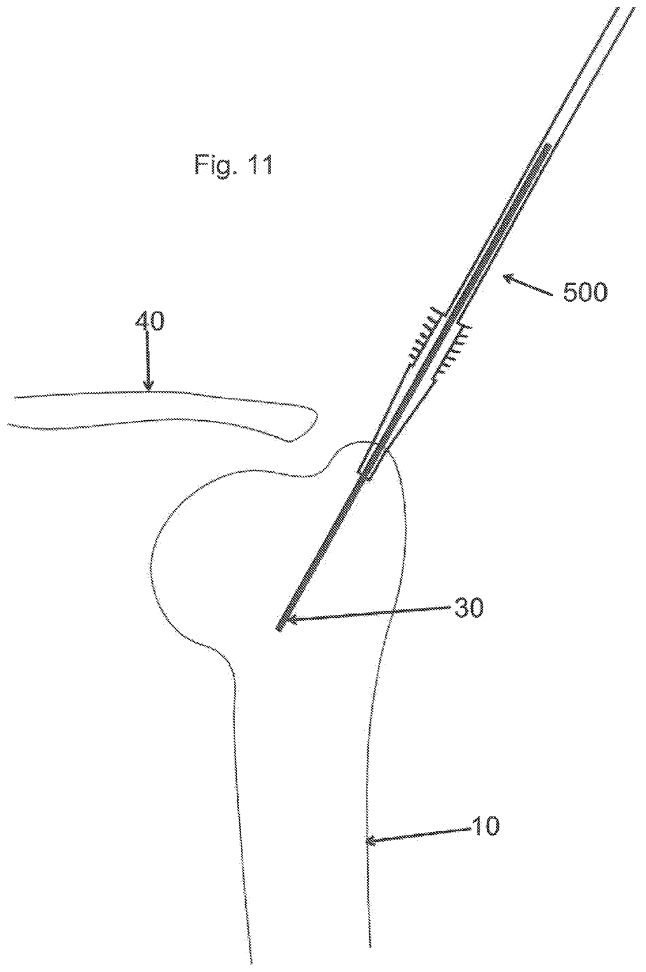

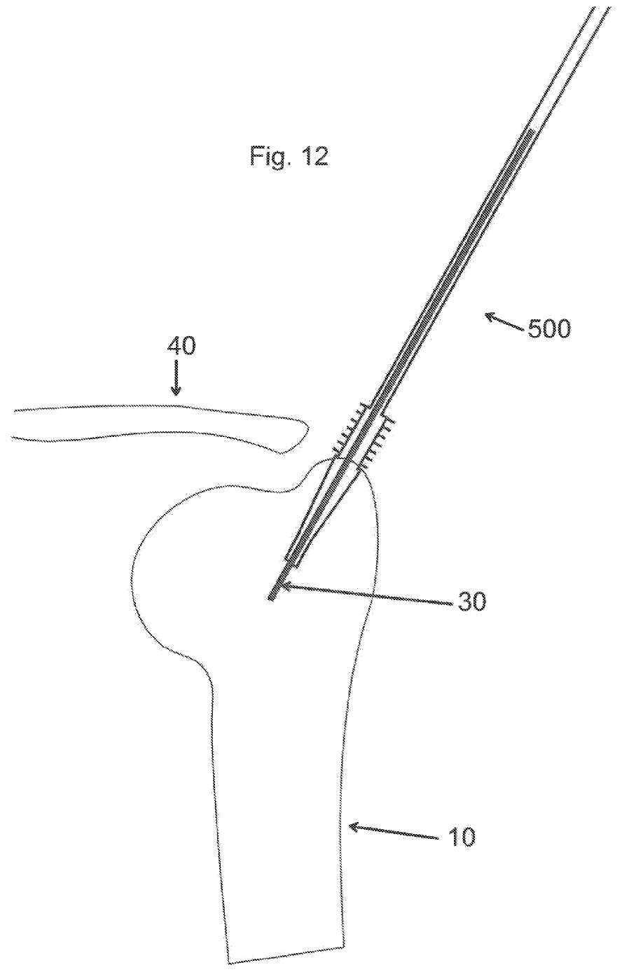

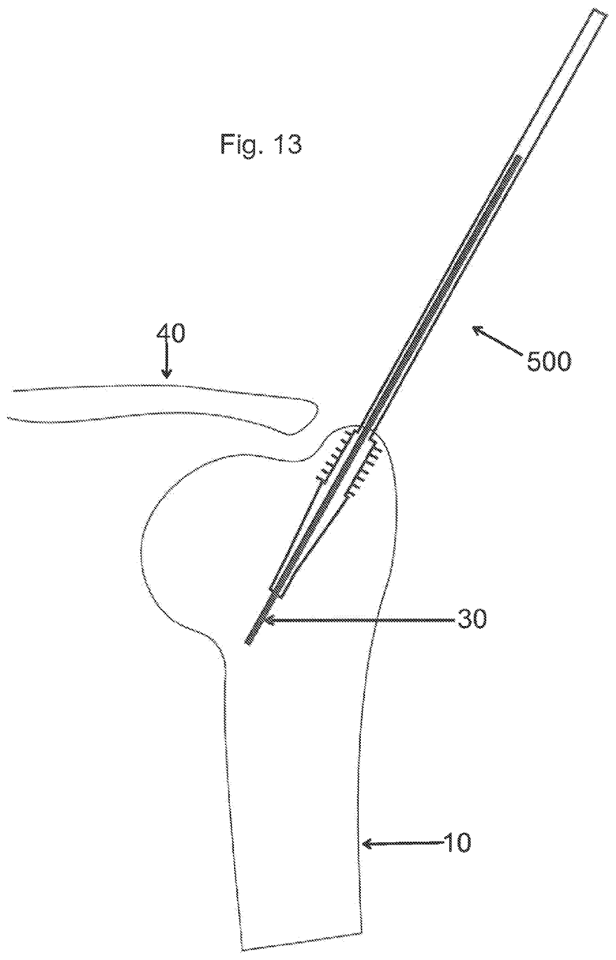

Int the preferred method described here, FIG. 11 illustrates the use of the drill bit 500 using a k-wire 30 to affect a partial drilling in the bone 10 for a soft bone density. FIG. 12 illustrates the use of the drill bit 500 using the k-wire 30 to affect a partial drilling in the bone 10 for a medium bone density. FIG. 13 illustrates the use of the drill bit 500 using the k-wire 30 to affect a drilling in the bone 10 for a hard bone density.

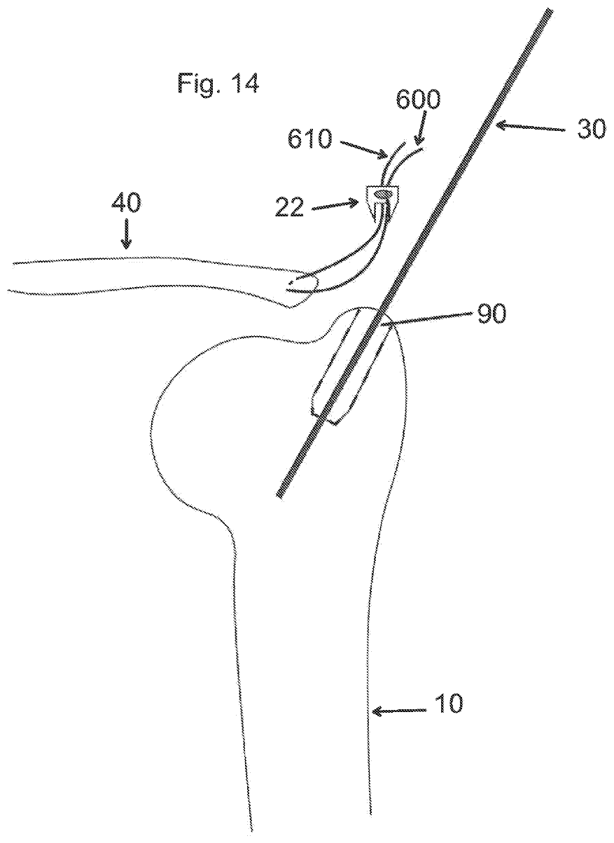

After the drill bit 500 is removed to create an opening 90 in the bone 10, at least one suture 600 is then coupled to the tendon 40 which is torn or detached from the bone 10. As illustrated in the preferred embodiment shown in FIG. 14, two of the at least one sutures (600 and 610) are coupled to tendon 40 and passed through two of the at least at least one tip suture passages entrances 122 and though the respective two at least one tip suture passages 120 of tip 22.

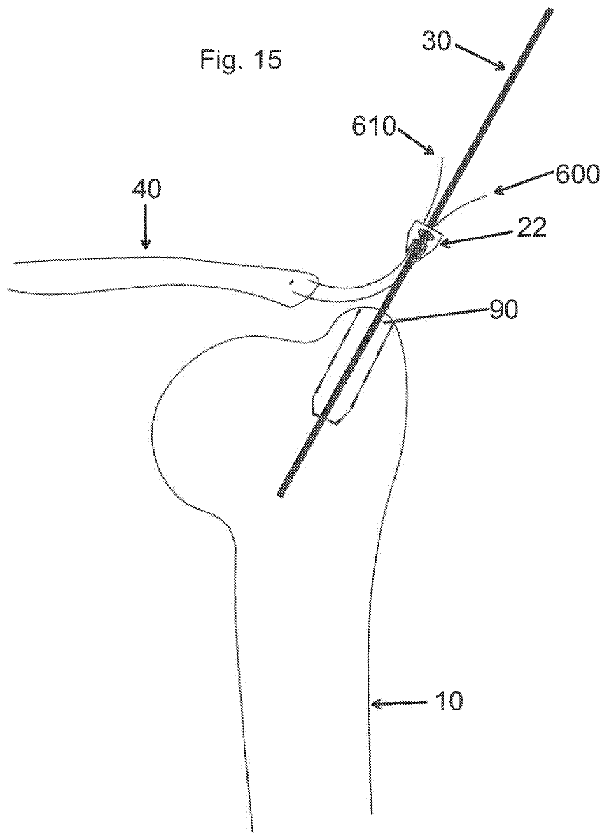

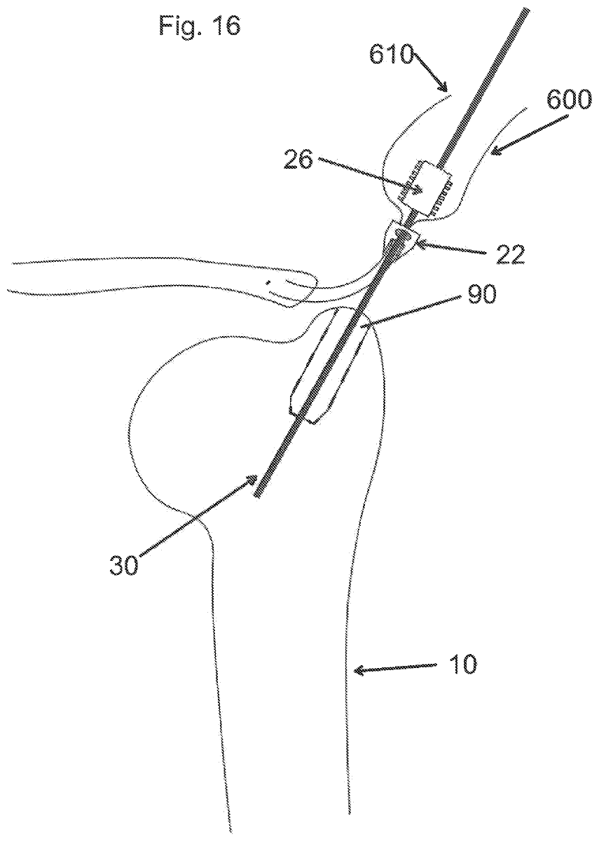

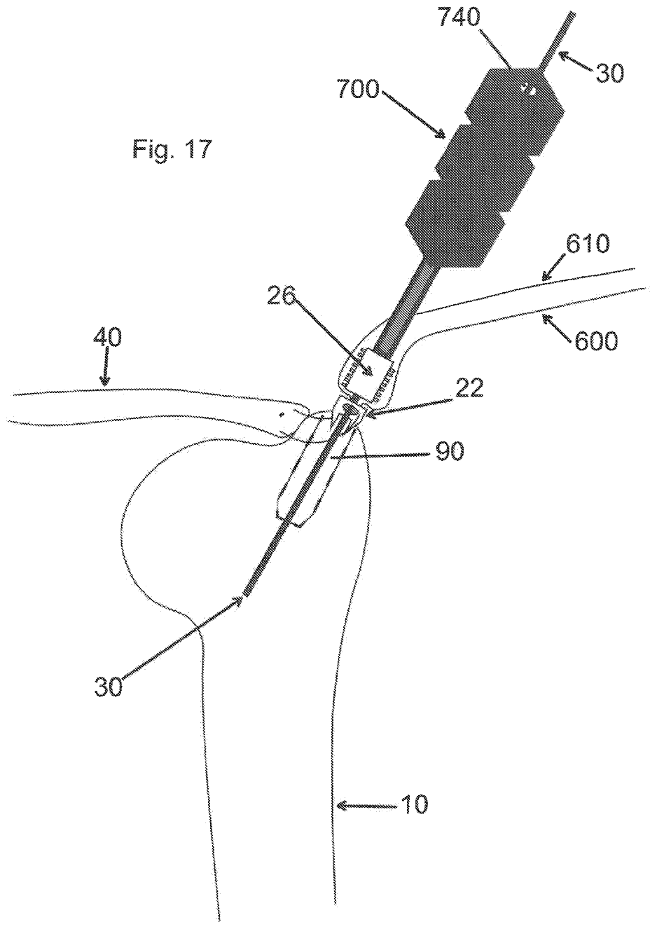

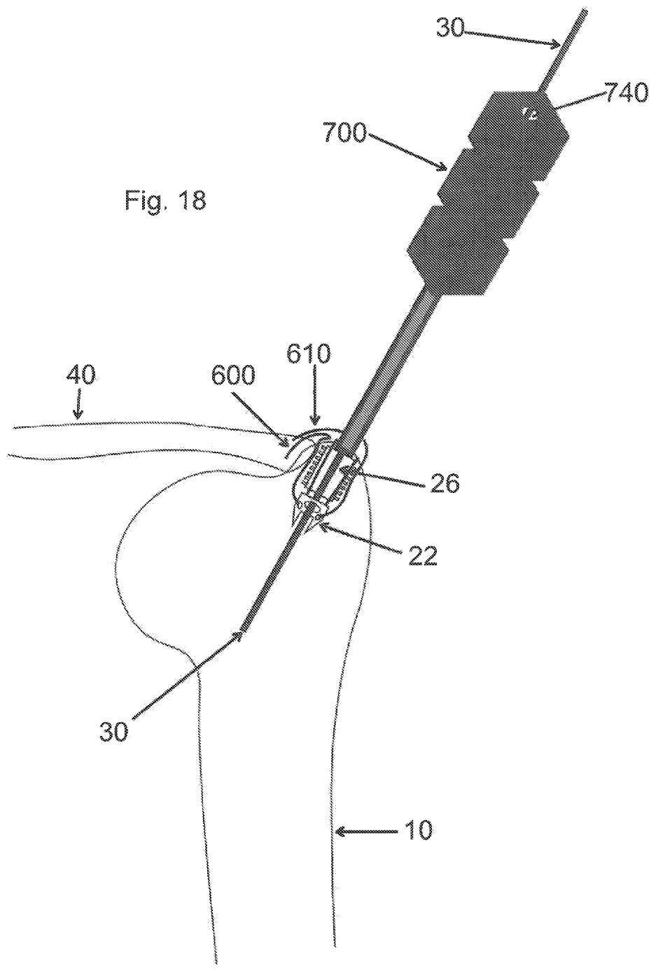

As illustrated in the preferred embodiment shown in FIG. 15, the tip 22 with sutures 600 and 610 is placed on the k-wire 30 using the tip k-wire passage entrance 110. As illustrated in the preferred embodiment shown in FIG. 16, the screw 26 is then placed on the k-wire 30 using the screw k-wire passage entrance 310. As illustrated in the preferred embodiment shown in FIGS. 17 and 18, a screw driver 700, preferably having a cannulated opening so that it may be placed on the k-wire 30, is then coupled to the screw 26. The screw driver 700 is then used to push and/or screw the screw 26 and the tip 22 into the bone which pulls the tendon 40 to the repair site of the bone 10 by tensioning the sutures 600 and 610. It should be noted that the screw driver 700 and the drill bit handle (not shown) may be may be the same device, which is the case for FIGS. 17 and 18.

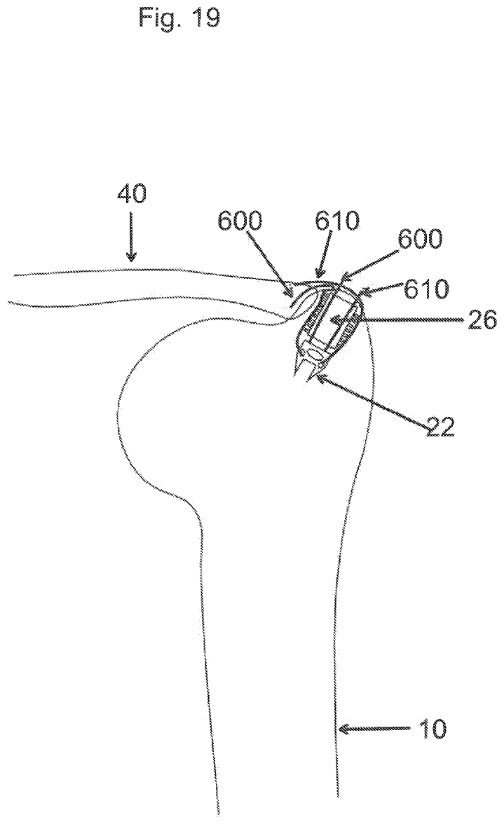

As illustrated in the preferred embodiment shown in FIG. 19, the screw driver 700 and the k-wire 30 are removed with any excess suture cut flush to the bone 10 surface.

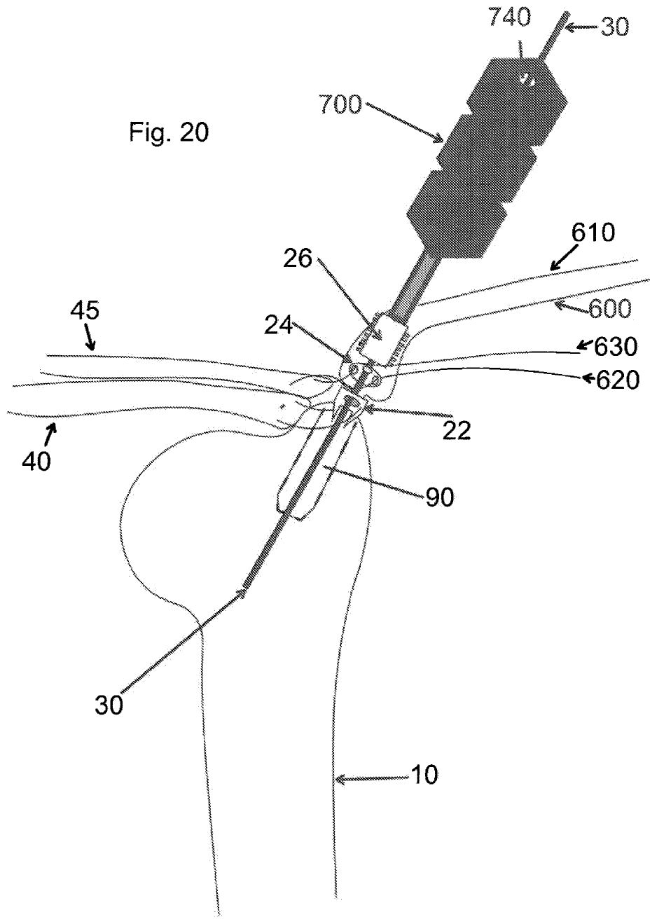

Another preferred embodiment is shown in FIG. 20, in addition to the tip 22 with sutures 600 and 610 being placed on the k-wire 30 using the tip k-wire passage entrance 110, another two of the at least one sutures (620 and 630) are coupled to another tendon 45 and passed through two of the at least at least one disc suture passages entrances 122 and through the respective two at least one disc suture passages 220 of the at least one disc 24 which are then placed on the k-wire 30 using the at least one disc k-wire passage entrance 210. Next, in this alternative preferred embodiment, the screw 26 is then placed on the k-wire 30 using the screw k-wire passage entrance 310 and the screw driver 700 is then coupled to the screw 26, preferably having a cannulated opening so that it may be placed on the k-wire 30. The screw driver 700 is then used to push and/or screw the screw 26, the at least one disc and the tip 22 into the bone which pulls the tendons 40 and 45 to the repair site of the bone 10 by tensioning the sutures 600, 610, 620 and 630.

The embodiments described above were chosen to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. An implant that is cannulated for anchoring a suture and used with a k-wire comprising:
a tip having a tip front, a tip central axis and a tip k-wire passage wherein the tip k-wire passage surrounds the tip central axis, and
an at least one disc: each at least one disc having a disc front, a disc back, a disc side, a disc central axis and a disc k-wire passage surrounding the disc central axis and an at least one disc suture passage separated from the disc k-wire passage, and
a screw having a screw back, a screw central axis and a screw k-wire passage wherein the screw k-wire passage surrounds the screw central axis, and
wherein the tip k-wire passage, the disc k-wire passage for each at least one disc, and the screw passage are aligned,
and
wherein the tip front is an implant front and the screw back is an implant back, and
wherein the implant from the implant front to the implant back includes, in order, the tip from the tip front to the tip back, each at least one disc each disc from the at least one disc front to the at least one disc back, and the screw from the screw front to the screw back.

14. The implant of claim 13 wherein each at least one disc suture passage includes:
a disc suture passage entrance selected from the group consisting of: the disc suture passage entrance extending to the disc front, and the disc suture passage entrance extending to the disc side, and the disc suture passage entrance extending to the disc back, and
a disc suture passage exit selected from the group consisting of: the disc suture passage exit extending to the disc front, the disc suture passage exit extending to the disc side, and the disc suture passage entrance extending to the disc back.

15. The implant of claim 14, wherein each the at least one disc suture passage is at least two.

16. The implant of claim 14, wherein each the at least one disc suture passage is at least three.

17. The implant of claim 14, wherein each the at least one disc suture passage is at least four.

18. The implant of claim 14, wherein each the at least one disc suture passage geometry does not include corners.

19. (canceled)

20. (canceled)
